(19) United States
(12) Patent Application Publication
(10) Pub. No.: US 2004/0049270 A1
(43) Pub. Date: Mar. 11, 2004

(54) Bone graft device

(76) Inventor: Robert J. Gewirtz, Bexley, OH (US)

Correspondence Address: Sean M. Casey Co., L.P.A., Attention: Sean M. Casey, P.O. Box 710, New Albany, OH, 43054-0710, US

(21) Appl. No.: 10/238,326

(22) Filed: Sep. 10, 2002

(51) Int. Cl. A61F 2/44

(52) U.S. Cl. 623/17.11; 606/61

(57) ABSTRACT

A bone graft device adapted to be received in an implant site in a patient, including, for example without limitation, an anterior spinal resection of one or more vertebrae of a spinal column of the patient. The bone graft implant site can be defined as a resection formed in any damaged or injured bone tissue, which can be, for further example, the spinal column resection that is formed between an inferior vertebral surface that confronts a superior vertebral surface where between one or more vertebral bodies and or vertebral discs or portions thereof have been removed to establish the implant site. The bone graft device includes a plurality of pseudo-vertebrae that are each formed with a transverse cross-sectional profile sized for implant in the anterior spinal resection. The pseudo vertebrae can be wedge-shaped and are stacked on at least one stanchion that can be centrally positioned or spaced apart. At least one of the plurality of pseudo-vertebrae defines an exteriorly facing sill that is adapted to be frictionally confronting and received against at least one of the inferior and superior vertebral surfaces to maximize frictional contact between the sill and at least one of the surfaces. Each pseudo-vertebral sill is sized and shaped to be equal to or smaller than the cross-section of the vertebral bodies defining the superior and inferior vertebral body surfaces so as to not invade the vertebral channel of the spinal column when the bone graft device is introduced into and received in the resection.

### TECHNICAL FIELD

This invention relates to the field of bone grafts. More specifically, the invention relates to a multimodal bone graft device that incorporates one or a plurality of bone pieces arranged in combination with one or a plurality of cortical bone pieces in new and novel configurations adapted for use in a variety of bone graft applications.

### BACKGROUND OF THE INVENTION

A normal human body has hundreds of bones. Collectively, these bones make up the skeleton and the skeletal system. This skeletal system has many functions in the body. One core function of the skeleton is to impart rigidity and definition to the human form. Another function of the skeletal system is to permit movement by acting as load-bearing members, levers, and as attachment anchors for muscles and connective tissues. The skeleton, although responsible for these and other structural functions in the human body, is itself also a dynamic, living organ system.

For example, red blood cells and most immune cells originate and mature in the marrow of bones in a process known to those with skill in the art as hematopoiesis. Furthermore, the mass of a human bone will vary from day to day as calcium stored therein leaches away from bone and into the blood stream and back again as the bone tissues continuously regenerate. Thus, in addition to establishing structural support and movement capabilities, bones also serve as an important reservoir for minerals and especially for calcium, which is an essential mineral that is required for muscle contraction, nerve impulse generation, nerve impulse propagation, and certain enzymatic functions.

The skeleton functions not only dynamically and mechanically as a collective system, but each skeletal bone is an individual tissue that may have a unique structure or function. Individual bones can vary greatly in their size, shape, density, thickness, perfusion, enervation, and other physical and physiological properties. A great deal of this variety stems from or contributes to the disparate functions performed by individual bones. There are many examples of such individual, specialized bone functions. For instance, the skull is actually formed from several smaller bones, that are joined together to protect the brain from damage. The rib cage and the spinal column similarly and respectively protect the thoracic organs and spinal chord, while the lesser bones of the ear facilitate hearing.

The bones of the feet are adapted to bear a much heavier weight and shock load than other bones such as those of the hand, which hand bones contrastingly move with greater degrees of freedom than the foot bones. The bones or vertebrae of the spinal column even have specialized functions within their ranks: the upper cervical vertebrae are especially adapted for twisting or rotation about a longitudinal axis so that the head can readily turn from side to side. In contrast, the lower thoracic or dorsal and lumbar spinal vertebrae are more limited in their range of relative motion while being capable of supporting much greater loads than the cervical vertebrae. The adaptation and specialization of various bones is not limited to the human animal; Pandas, for example, use one of their wrist bones as an opposable digit, a pseudo thumb of sorts.

To accomplish effectively these diverse functions and tasks, many bones have specialized features. One obvious example of such specialization is the shape of the bones. The skull, the tibia, and the sternum look nothing like each other, because each of these bones is defined to be functionally and geometrically disparate and to accommodate different tissues, organs, and capabilities. In addition to bone shape being highly variable, there is also considerable variation in the cross-sectional density of each bone. Most bones have a generally sponge-like or cancellous interior body portion that while being substantially rigid is less dense that an outer, more dense, and compact layer that is referred to as the cortical rim or layer. Depending upon the load and shock bearing requirements of a given bone, this outer shell can have a widely varying thickness. This outer cortical layer or shell or rim imparts much of the strength to the bone. Relatively thick cortical shells or rims are typical of, for example, the vertebrae of the spinal column, as well as for the bones of the legs, and other bones that carry a high weight or compressive or tensile load. In contrast, the flat bones that are the site of hematopoiesis tend to have relatively increased amounts of the spongy, inner layer of bone known to those having skill in the art as the cancellous layer or cancellous bone. The porosity of the cancellous layer provides an incubation or living space for the developing blood and immune system cells, while the increased perfusion that is typical of cancellous bone brings adequate supplies of food, nutrients, and other factors to these developing cells.

In spite of the diversity of the form and function of various bones, all bones have substantial physiological similarities to each other. For example, bones are typically composed of the same essential materials, regardless of specialized function or shape. A significant portion of bone is organic material, mostly protein-associated glycosaminoglycans and especially collagen, a protein commonly found in connective tissues and in extracellular matrixes. About half of the bone mass is mineral, and the most common bone-associated mineral is a calcium compound that closely resembles hydroxyapetite. Like the rest of the body, bone also contains a significant amount of water.

In addition to composition, bones dissimilar in shape and size usually share other physiological similarities. For example, a thin layer of connective tissue typically covers the outer surface of the bone. Similarly, a thin, membranous layer lines the interior cavity of the bone. This thin, membranous layer is associated with several bone-producing cell types such as osteogenic cells, osteoblasts, and osteoclasts, for example. Those with skill in the art may know these two layers as the periosteum and the endosteum, respectively.

Another example of physiological similarities shared by various types of bones is vascularization. Blood vessels penetrate and permeate the bone through a series of channels and canals. These channels and canals can vary in location, length, and diameter. Examples of such channels include the canaliculi, haversian canals, osteons, Volkmann's canals, and others. These canals bring blood, nutrients, and other vital factors to and carry away waste from the multitude of cells that live in and that form the bone. One important example of such a bone-dwelling cell is the pluripotent stem cell that can differentiate into and form any of the cells of the blood or immune system. The blood and immune cells themselves also reside, at least for a time, in the bone. Such cells include macrophages, neutrophils, B cells, various T cells, eosinophils, basophils, megakaryocytes (the progenitor of platelets), and red blood cells. There are also varieties of bone-forming cells such as the osteoclasts, osteoblasts, and osteogenic cells mentioned above that live in the bone. Attached to the bones may be various tendons and other connective tissues that cooperate with the bones and any connected muscles to establish the articulation of movement or to secure tissues or organs in place.

Yet another example of shared bone physiology is the specialized structures located at one or both ends of some bones. These specialized structures are adapted to hold a bone in a joint, while at the same time giving some degree of freedom and relative motion to the jointed bones. Examples of this type of arrangement include the bones that form the elbow, knee, shoulder, finger, ankle, foot, vertebral, and similarly movable joints. In addition to these and other physiological similarities, bones also can share developmental similarities. For example, the process of either intramembraneous ossification or endochondral ossification forms the bones, wherein the latter process involves a cartilaginous intermediate that is calcified to form the resulting bone tissue. Fibroblast cells typically lay down a network of collagen fibrils, upon which are deposited the calcium crystals in a mineral form approximating hydroxyapetite.

Aggregates of such calcified fibrils form fibers that then form the bone. The arrangement of the fibers can significantly affect the gross anatomy and function of a bone. For example, fibers can be substantially parallel to each other, perpendicular to each other, may be in a stochastic order, or some combination thereof. The fibers may be arranged in lamellae or can be interwoven. The density of the fibers can also affect the properties of bone. The fibers can be dense and compact, such as in the tough outer rim, shell, or layer of cortical bone, or the fibers can be loosely arranged and spongy, such as in the inner body of cancellous bone tissue. Further details of the preceding discussion as well as additional information about bone development and physiology can be found in the fourth chapter of the Color Atlas of Histology, second edition, by Leslie P. Gartner and James L. Hiat, Williams & Wilkins, Baltimore, Md., USA, 1994.

As can be understood by those skilled in the art, the complexity of bone functions and structures establish a skeletal system having extraordinary capabilities. Yet, as with any highly-refined and precisely engineered system, many possible anomalies can develop and afflict the system that can include disease, degeneration, and failure. Developmental disorders caused by genetic diseases such as osteogenesis imperfecta can cause imperfectly formed or non-functional bones. Even if bones develop correctly, bones can break and fracture and can be susceptible to infections and diseases such as Staphylococcus aureus, which can kill an otherwise healthy bone, possibly necessitating resection, removal, and amputation. Osteoporosis, a degenerative disease common among woman as they age, can leave bones brittle and easily broken. Conditions such as spondylolisthesis, pinched or compressed spinal nerves, and degenerative and stenosis inducing intervertebral disc diseases can all cause pain and limitations on possible range of motion. In addition to diseases and disorders, trauma such as that caused by automobile accidents, falls, collisions, heavy loading, and the like can also damage or break a bone and can injure connective and interstitial tissues.

Often, it is possible to correct or limit such maladies. Contagious diseases frequently respond to antibiotic treatment. Diet, exercise, medicaments, and other therapies can sometimes treat or control other disorders. Bone fractures that result from trauma can be routinely set, which term “set,” among others, is a term of art that describes the process of holding the broken ends of bone together long enough for them to grow back together. Casts, pins, screws, and braces can also be employed to treat injured or damaged bones. Even with the myriad treatment options available to the patient and practitioner for treating bone diseases and disorders, such treatments alone can be sometimes insufficient or inappropriate. In some cases, additional treatment regimes are indicated and can include resection and removal of damaged bone and connective tissues and grafting of corrective and replacement structures and tissues. Bone grafting can be particularly effective in repairing damaged structures and can include autografts wherein bone is removed from a healthy bone structure in the patient and transplanted to replace damaged bone tissue that has been removed. Allografts can also be obtained from a bone bank and used in similar fashion.

When performing a graft, a surgeon or health care practitioner may have several goals and objectives in repairing an anomalous or injured bone structure. Such goals can include speeding recovery, healing, and regeneration of replaced tissues, as well as reducing the risk of infection and minimizing post-operative pain, are common to many other surgical procedures. The primary benefit of bone grafting in accelerating healing and regeneration of replaced bone tissue, is that the graft establishes an in vivo template upon which the body can build new bone. The inserted bone graft, while providing interim support during the post-operative healing process, is not the final structural repair to the bone; rather it is a scaffolding of sorts.

If the procedure is successful, the body will deposit calcium, connective, and other tissues onto the implanted graft or scaffold. Over time, living bone will be formed around the inserted piece of bone or graft. Eventually, the graft will be fused into the recipient bone to form one continuous bone. As time continues to pass, eventually the entire graft will be completely scavenged and replaced with regenerated bone in the same way as all bone tissue is continually regenerated. As is known to those skilled in the various related arts, the removal of damaged or injured bone and surrounding tissues exposes extant bone surrounding the site of the bone graft, which signals the proximate and remaining healthy bone that damage exists. The body responds by growing new bone around the site into the template bone graft. This process mimics the response to, for example, a broken bone that fuses back together following the break or facture.

Often, another of the principle aims of the graft is to restore structural integrity and function to a limb, the spine, or other repaired bone tissue. In this case, the practitioner often intends for the graft itself to provide some structural support to the surrounding bone structure immediately upon implant and throughout the subsequent healing, recovery, and regeneration period. The structural support increases as new bone deposits around the graft. A further objective of bone grafting, particularly in the example of back or spinal column procedures, may be to relieve nerve root compression, reverse stenosis, and to replace damaged vertebral and disc elements, which can in turn reduce or ideally eliminate pain. Such spinal procedures can also further include, for example, the fusion of lower back vertebrae to alleviate pain and discomfort. In other instances, another aim of the bone graft procedure may be cosmetic in nature, such as the graft-mediated reconstruction of a jaw or cheekbones.

To help accomplish these and other surgical objectives, several permutations of grafting are possible. In one embodiment, a surgeon removes tissue from the patient and then transplants the tissue in a new location on that same patient. Such a graft is often referred to as an autograft to those with skill in the art. Donor material obtained from another individual that is genetically identical to the recipient is often referred to as an isograft, which is mostly just as preferable as an autograft. A surgeon might perform an auto- or isograft on, for example, a burn victim. In the case of a burn victim, the surgeon can remove undamaged and or donor skin from the back, buttocks, or another inconspicuous area and then the surgeon can graft that skin onto a burned or otherwise badly damaged area of skin. Autografts and isografts such as skin grafts offer several advantages over other forms of graft. One important advantage is a decreased chance of disease transmission; since the patient receives their own tissue they are not likely to contract any new diseases from that tissue. Another important advantage of an auto- or isograft is that immunological rejection of the tissue is not an issue, since in a healthy person the immune system tolerates its own tissues. An additional advantage of a bone auto- or isograft is that the fresh, living bone readily supports the deposition of new bone and therefore increases the chances of a successful graft procedure.

In spite of these and other advantages, it is often not possible, convenient, or desirable for the surgeon to perform an auto- or isograft. This limitation is especially pronounced in the case of a bone auto- or isograft, where a surgeon excises a piece of bone from a healthy bone such as the pelvis proximate to the iliac crest and then implants that piece of bone elsewhere in the patient. This procedure usually requires two incisions and therefore exposes the patient to twice the risk of surgical infection. The site from which the bone is removed can be very painful during recovery and can result in restricted mobility. Furthermore, it may be impossible to remove a suitably sized or dimensioned bone piece without compromising the integrity or function of the donor bone. As a result of such limitations, surgeons often perform a second type of graft known to those with skill in the art as an allograft.

An allograft is substantially similar to an autograft or isograft, except that the allograft is obtained from another individual of the same species. In the case of a bone allograft, the donor is usually a recently deceased cadaver. One significant advantage of using cadaver bone is that the surgeon can harvest any piece of or all of a bone, since there are no concerns of maintaining the structural or functional integrity of the donor bone. Furthermore, while cadaver bone is readily available and relatively easy and inexpensive to acquire, acquiring the precise size, shape, and density of such allograft material from a bone bank can often be extremely challenging.

There are further limitations associated with such allografts. Although proper handling can mitigate concerns, one important limitation is the possibility of disease transmission from the cadaver to the allograft patient. In addition to infectious disease, the recipient body may reject the foreign or allograft tissue, which can lead to further complications. Furthermore, the size, density, and other physical features of a bone can vary considerably from person to person, and depend upon the donor age, health, and other factors.

Since the graft implant site of the patient is usually not prepared and defined until the procedure is underway, the surgeon must compensate or adjust for the variability of available bone autograft or allograft materials during the surgical procedure. At that time, it may also be difficult for the surgeon to obtain a piece of bone that is exactly the right size and shape or that has the proper load bearing capacities. Accordingly, the available bone graft material must be further modified before being implanted into the patient. The load bearing capacity of the available bone graft material is especially relevant to grafts to be effected in, for example, the bones of the leg or the spinal column. While cortical bone grafts will impart the needed strength to spinal or other high load bearing grafts, cancellous bone is more likely to encourage deposition of new bone so as to improve the likelihood of a successful graft and rapid recovery. Those skilled in the art have often amplified concerns that it is time consuming, difficult, and often impossible during the procedure to obtain an autograft or allograft bone piece of the most desirable size and with the most desirable ratio of cortical and cancellous bone that will optimize the strength and regeneration capabilities of the bone graft.

In addition to the iso- or autograft and allograft, another general type of graft is the xenograft. The xenograft is substantially similar to the allograft, except that the donor is of a different species than the recipient. The xenograft offers the advantage of a potentially plentiful supply of relatively inexpensive tissue. However, the xenograft shares many limitations with the allograft such as the risk of disease transmission and immune system rejection. In addition to the limitations that the xenograft shares with the allograft, the xenograft is only possible with those tissues that are compatible across different species. There are also ethical considerations involved when using or contemplating animal tissue as a substitute for human tissue. Because of these serious limitations, surgeons typically perform xenografts only in very limited situations.

Such bone grafts can be employed alone or in combination with other non-biological materials, instruments, and devices. Such non-biological grafts can incorporate or can replace auto- and allografts and can include, for example, artificial or synthetic materials and devices. In theory, non-biological materials, instruments, and device can avoid the limitations of disease transmission, immune rejection, and limited supply or availability of autografts and allografts. For example, surgeons sometimes use an artificial skin that is grown in a laboratory from one or a plurality of cell types, in place of skin auto- or allografts.

This technology has limited applicability, however, because the implanted material needs to perform many or most of the same structural and physiological functions as the tissue that is replaced. Furthermore, the material must be both biocompatible and stable under physiological conditions that can be extremely harsh. These limitations also apply to artificial bone grafts, instruments, and devices. As discussed above, bone tissue is not only a mechanical structure but is also a complex and diverse combination of living, dynamic, and continuously regenerating tissues. Science can provide materials that mimic the structural properties of bone, but it has yet to discover a material that also duplicates its extraordinary biological properties and capabilities. Artificial bone materials do not always or in every patient readily encourage or support the deposition of new, living bone and are therefore not the always the preferred materials for bone grafts in every patient or in every malady.

With the preceding considerations in mind, those having knowledge in the art may comprehend that there are many considerations relevant to a determination of the most appropriate graft material and technique. If the patient and doctor elect an autograft approach, a suitable piece of bone must be excised from the patient before or during the graft procedure. On the other hand, if an allograft is preferred, a suitable donor bone piece, usually from a cadaver, must be ordered from a bone bank before the procedure is commenced. Regardless of which type of graft is elected, or even if both are needed, it may also be necessary to employ one or more artificial materials, instruments, and or devices to augment the graft procedure.

Even with the best and most thorough of pre-operative diagnoses and analyses, the surgeon typically can only define all of the size, shape, and strength parameters of the desired graft, whether it be allograft or autograft bone material, or artificial materials or components, or some combination thereof, once the damaged or injured tissues are visually inspected and resected and prepared for the graft during the procedure. If an autograft bone graft is to be used from the patient, the autograft materials is usually prepared at the same time by excising material from a donor bone such as a portion of the iliac crest or from the bone tissue being removed from the location of damage or injury. If an allograft bone piece is to be employed in the procedure, it must usually be modified before implant so that it can be properly sized and dimensioned to fit the space established for the graft after the original tissues are removed. Moreover, even if a detailed pre-procedure analysis establishes suitability for various artificial articles, such items are often modified during the procedure to accommodate specific anatomical variations only evident upon visual inspection and after preparation of the graft location.

Several attempts have been made to offer improved methods and devices that address some aspects of past problems and difficulties. For example, O'Leary et al. in U.S. Pat. Nos. 5,073,373 and 5,484,601 are restricted to teaching, among other limitations, a bone powder that is includes cortical and or cancellous bone constituents that are reconstituted into implantable pastes and cakes. The O'Leary et al. bone powder may be suspended in glycerol, polyhydroxy compounds, or the like prior to being implanted. The O'Leary et al. approach has been attempted in many variations and also by others and all such attempts demonstrate several shortcomings that include the need for potentially immunogenic and inflammatory materials like animal collagen and glycerol, which can have adverse consequences. Even without the possible consequences, the contemplated materials and compositions offer little to no immediate structural support that can alleviate symptoms such as stenosis and other sources of never root compression indicated in various spinal column anomalies.

Other examples in the prior art that are restricted to mechanical, bone powder-retaining devices such as a cage are described in U.S. Pat. No. 5,489,308 to Kuslich et al. and U.S. Pat. No. 5,514,180 to Heggeness et al. The approaches advocated by Kuslich et al. and Heggeness et al. fail to, among other problems, establish an effective means to ensure that the graft instrument fuses into the host bone. Even though various elements are included that purport to facilitate such fusion, the suggested '308 and '180 instruments have only limited application since they cannot be modified to accommodate newly visualized in vivo size and shape parameters during a procedure without remachining of the contemplated material of the implant at the time of the implant, which defeats the preconfigured nature of the devices. Moreover, the '308 and '108 implant devices also will likely be subject to long-term displacement as the surrounding host bone tissue undergoes continual regeneration, which process can change the dimensions and profiles of surrounding host surfaces that can destabilize and loosen the implant thus necessitating another surgical procedure. U.S. Pat. No. 5,910,315 to Stevenson et al. is similarly limited to combinations of the preceding devices wherein a metal cage is received into a bore in the host bone and is injected with a powered bone material. Here again the metal cage can be unseated and destabilized over time as the host tissues regenerate and the metal cage is not easily modified during the procedure to accommodate newly prepared and or visualized implant site size, shape, and load bearing requirements and parameters.

Still another approach is to use machined, prefabricated graft compositions that, in whole or in part, are bone tissue. Such graft compositions may be ground, compressed cancellous bone, cortical bone, or combinations thereof. The graft compositions can be shaped and machined into various shapes and sizes that can potentially accommodate a variety of graft or implant sites and environments. An example of the prior art that teaches this type of approach is U.S. Pat. No. 6,371,988 to Pafford et al., which is limited to a graft material that is treated with compounds or proteins that can enhance the capability to act as a template for the deposition of new bone. Although this approach may address some of the limitations of the prior art, the surgeon has to choose correctly the size and shape of the graft before surgery. Another significant limitation of the '988 approach is that the surgeon is burdened with the need take time to precisely shape and mold the prefabricated '988 graft during the procedure. The very type of modifications likely can eliminated many of the purported useful features of the Pafford et al. device before being implanted. Moreover, the various limitations that Pafford et al. require can significantly increase the difficultly in obtaining suitably configured graft devices prior to the procedure, can increase the cost thereof, and will reduce the available supply of such bone graft material because of the tremendous loss of material incurred to fabricate the intricate features of the '988 device.

In attempts to avoid complications resulting form the use of ground bone as a graft material, artificial or synthetic bone grafts have been described in the art such as those in U.S. Pat. No. 5,258,043 to Stone, U.S. Pat. No. 4,904,260 to Ray et al., and U.S. Pat. No. 5,626,861 to Laurencin et al. Each of these attempts is restricted to, among other elements, the use of prosthetic implant material, in various embodiments that are purportedly aimed at instigating improved rehabilitation of host vertebral bone and disc tissues. While the contemplated artificial or synthetic materials can be more easily supplied than other bone graft materials, the noted synthetics suffer from the pitfalls that they do not function in vivo in the same way that actual bone or bone-derived tissue functions with respect to structural integrity, load bearing capacity, fostering new bone deposition, and the like.

Yet another attempt to overcome the limitations of the prior art are illustrated in U.S. Patent Publication No. US 2002/0029084 to Paul et al. The teachings of Paul et al. are limited in many respects to the various methods and devices already discussed and contemplated hereinabove and fail in all respects to address all of the most troublesome difficulties the plague the art. The Paul et al. devices are restricted to, among other limitations, hybrid cortical and cancellous grafts having fixed predetermined shapes, sizes, and dimensions that are not readily reconfigurable during a procedure to easily accommodate the newly visualized, prepared, and possibly unusually configured implant site in a way that overcomes in any way the many shortcomings of the many prior art devices and methods.

What has long been needed but heretofore unavailable is a readily obtainable, relatively inexpensive, easily reconfigured, structurally useful, and rapidly assimilable bone graft device and bone graft kit that is suitable for use as an implant in a variety of bone graft procedures. The preferred method or device must reduce the burden on the surgeon during a selected procedure while maximizing the options available for preparing and presenting a bone graft implant that incorporates the most desirable shape, size, and dimensions for a given implant site. While being consonant with established medical practice, and while having wide compatibility with conventional surgical procedures, the desired bone graft should be especially well-suited for the most complicated procedures including, for purposes of example without limitation, corpectomies, discectomies, and other similarly complex reconstructive and rehabilitative procedures.

### SUMMARY OF INVENTION

In its most general configuration, the present invention advances the state of the art with a variety of new capabilities and benefits while and overcoming many of the shortcomings of prior devices in inventive and novel ways. In one of the many preferable configurations, a medical practitioner selects an appropriate number of cancellous bone pieces based upon the desired size of the graft. The practitioner, after removing damaged bone and tissue and preparing an implant site then assembles the bone graft device using one or more specially configured cortical bone pieces and surgically implants the graft to help to repair damage to bone caused by trauma, disease, or the like.

In one of many variations of the instant invention, the bone graft device is to be received in a prepared graft implant site in a patient. Such an implant site can be in any of a number of bone graft site locations such as, for purposes of example but not for limitation, an anteriorly approached spinal resection of one or more cervical, thoracic (or dorsal), or lumbar vertebrae of a spinal column of the patient. The bone graft implant site can be defined as a resection formed in any damaged or injured bone tissue, which can be, for further example, the spinal column resection that is most commonly referred to by those skilled in the relevant arts as a corpectomy wherein one or more vertebral body portions and intermediate vertebral discs are partially removed so that an inferior vertebral surface confronts a superior vertebral surface to establish the implant site.

The bone graft device preferably incorporates a plurality of pseudo-vertebrae that are each adapted with at least one transverse cross-sectional profile that is sized to be compatible for receipt and implant in the anterior spinal resection. The pseudo vertebrae can be wedge-shaped about a sagittal and or coronal cross-section and are preferably configured to be stacked end-to-end on one or more struts or stanchions, which can positioned in a generally central aperture or in a multiple stanchion spaced apart configuration. At least one of the plurality of pseudo-vertebrae is formed with an exteriorly facing sill that frictionally confronts and is received against at least one of the inferior and superior vertebral surfaces. As best as possible, the sill surface is adapted with to maximize frictional contact with the at least one of the vertebral surfaces. To avoid invading the vertebral channel or foramen of the spinal column, each pseudo-vertebral sill is sized and shaped to be, when introduced into and received in the resection, equal to or smaller than the transverse or horizontal cross-section of the vertebral bodies, which bodies define the superior and inferior vertebral body surfaces.

Additional modifications of the preferred embodiments may optionally or preferably incorporate at least one of the plurality of the pseudo-vertebrae to have a generally elongated cardioid cross-sectional horizontal or transverse profile. The cardioid profile can be proximal to the exteriorly facing sill such that the profile is approximately smaller than and for the most part is substantially circumscribed by the confronting proximate vertebral surface that is received against the sill.

A further variation of any of the preceding embodiments may also include the bone graft device being modified wherein at least one of the plurality of pseudo-vertebrae is formed to have a substantially wedged shaped cross section about a sagittal or coronal plane. In this configuration, the bone graft device can incorporate the exteriorly or outwardly facing sill to be more closely coplanar with and contacting the confronting vertebral surface, which vertebral surface can be, after the implant site has been prepared, somewhat oblique relative to a generally horizontal or transverse plane or section cut plane.

Any of the preceding configurations and embodiments may also be adapted to include any one of a number of the at least one pseudo-vertebra that can optionally or preferably be adapted to rotate, as the bone graft device is implanted into and introduced into the resection. The at least one pseudo vertebra can rotate about an axis that is substantially parallel with an axis of the at least one stanchion, and which stanchion and rotation axis can be substantially or approximately parallel with a substantially superior to inferior longitudinal hypothetical line that defines an intersection of a sagittal and a coronal plane. In this modified embodiment, a superior abacus pseudo-vertebra can rotate relative to a sandwiched drum pseudo-vertebra and an inferiorly stacked plinth pseudo-vertebra can similarly rotate relative to the drum. With this rotational capability, the superior abacus and the inferior plinth pseudo-vertebrae can, if also somewhat wedge-shaped, realign their respective exteriorly or outwardly facing sill surfaces to more closely be nearly or substantially coplanar with the confronting vertebral body surfaces against which the sill surfaces are received as the implant is introduced into the implant site.

While rotation of the super-most (abacus) and inferior-most (plinth) pseudo-vertebrae may be optionally preferably, the one or more drum or intermediate pseudo-vertebrae can incorporate one or more key elements that are configured to prevent relative rotation between the one or more drum pseudo-vertebrae during and after implantation. Instead of or in combination with such key elements, the bone graft device may also be further optionally or preferably modified to wherein one or more of the key elements cooperate with at least one strut or stanchion key, keyway, or corresponding key element that may be formed in the at least one stanchion. The at least one stanchion key, keyway, or corresponding key element may be modified to prevent relative rotation between the at least one stanchion and the one or more drum pseudo-vertebrae.

The preceding embodiments and configurations may also be further configured to incorporate the one or more struts or stanchions that may be partially curved about an axis passing longitudinally therethrough from a superior end to an inferior end such that the lordotic or kyphodic curves of the spinal column can be more closely approximated with the assembled bone graft device. In yet additional modifications to any of the preceding arrangements, the bone graft device may incorporate the plurality of pseudo-vertebrae to be received about at least two stanchions whereby the pseudo-vertebrae are thereby keyed to prevent relative rotation between the pseudo-vertebrae. Each of the one or more struts or stanchions may be centrally or peripherally arranged when received with the pseudo-vertebrae to accommodate any of a number of possibly desirable structural configurations.

Any and or each of the preceding configurations and embodiments may also be adapted wherein the various bone graft device components, elements, and features are included in a kit that incorporates of plurality of variously configured, sized, shaped, dimensioned, and modified pseudo-vertebrae, struts or stanchions, and similar components that can be alternatively formed from isograft, allograft, and autograft bone pieces including cortical, cancellous, hybrid, as well as artificial materials, and combinations thereof.

These variations, modifications, and alterations of the various preferred embodiments may be used either alone or in combination with one another as can be better understood by those with skill in the art with reference to the following detailed description of the preferred embodiments and the accompanying figures and drawings.

### BRIEF DESCRIPTION OF THE DRAWINGS

Without limiting the scope of the present invention as claimed below and referring now to the drawings and figures, wherein like reference numerals, and like numerals with primes, across the several drawings, figures, and views refer to identical, corresponding, or equivalent elements, components, features, and parts:

FIG. 1 is an elevated perspective anterior view illustrating a bone graft device according to the principles of the instant invention in a pre-inserted and a seated position in vivo in a resection implant site of a cervical spinal column of a patient;

FIG. 2 is an elevated perspective oblique lateral or sagittal view, rotated and in reduced scale, of the bone graft device of FIG. 1, and with a portion of a thoracic or dorsal spinal column of a patient set apart for purposes of illustration;

FIG. 3 is a sinistral sagittal cross-sectional view, rotated and in modified scale, of a variation of the bone graft device of FIGS. 1 and 2;

FIG. 4 is a sinistral sagittal cross-sectional view, rotated and in modified scale, of another variation of the bone graft device of FIGS. 1, 2, and 3;

FIG. 5 is an elevated perspective view, in modified scale, of a pseudo-vertebra of the bone graft device of FIGS. 1 through 4 according to the principles of the instant invention;

FIG. 6 is an elevated perspective view, in modified scale, of a stanchion or strut of the bone graft device of FIGS. 1 through 4;

FIG. 7 is an elevated perspective assembly view, in modified scale, of the bone graft device of FIGS. 1 through 4;

FIG. 8 is an elevated perspective assembly view, in modified scale and with a portion of the device cut away for purposes of illustration, of the bone graft device of FIG. 7;

FIG. 9 is an elevated perspective view, in modified scale, of the stanchion or strut of FIG. 6;

FIG. 10 is a cross-sectional view, in modified scale, rotated and taken about section line 10-10 of FIG. 9;

FIGS. 11 through 13 are cross-sectional views, in similar scale, rotated and which could have taken about a section line similar to that of section line 10-10 of FIG. 9 of variations of the stanchion or strut of FIGS. 6 and 9;

FIG. 14 is an elevated perspective view, in similar scale, of a variation of the stanchion or strut of FIGS. 6 and 9;

FIG. 15 is an elevated perspective view, in a similarly depicted scale, of the pseudo-vertebra of FIG. 5;

FIGS. 16 through 20 are elevated perspective views, in similar scale and rotated, of variations of the pseudo-vertebrae of the FIGS. 5 and 15;

FIG. 21 is an elevated perspective view, in similar scale, of a variation of the pseudo-vertebra of FIG. 17;

FIG. 22 is a superior transverse or horizontal view, in similar scale and rotated, of the pseudo-vertebrae of FIGS. 5 and 15;

FIG. 23 is a cross-sectional view, rotated, in similar scale, and taken about section line 23-23 of FIG. 22;

FIG. 24 is a cross-sectional assembly coronal or sagittal view, in similar scale, of multiple and stacked pseudo vertebrae of FIGS. 21 through 23;

FIG. 25 is an elevated superior perspective view, in similar scale, of a variation of the pseudo-vertebra of FIG. 20;

FIG. 26 is an elevated inferior perspective view, in similar scale, of the pseudo-vertebra of FIG. 25;

FIG. 27 is a superior transverse or horizontal view, in similar scale and rotated, of the pseudo-vertebrae of FIGS. 20, 25, and 26;

FIG. 28 is a cross-sectional view, rotated, in similar scale, and taken about section line 28-28 of FIG. 27;

FIG. 29 is a cross-sectional assembly anterior coronal view, in similar scale, of multiple and stacked pseudo vertebrae of FIGS. 25 through 28;

FIG. 30 is an elevated perspective superior view, in similar scale, of a variation of the pseudo-vertebrae of FIGS. 17, 21, and 25;

FIG. 31 is an elevated perspective superior view, in similar scale, of a variation of the pseudo-vertebrae of FIGS. 5, 15, 22, 23, and 24;

FIG. 32 is a cross-sectional view, rotated, in similar scale, and taken about section line 32-32 of FIG. 31;

FIG. 33 is a cross-sectional assembly sagittal or coronal or oblique view, in similar scale, of multiple and stacked pseudo vertebrae of FIGS. 31 and 32;

FIG. 34 is a cross-sectional assembly sagittal or coronal or oblique view, in similar scale, of multiple and stacked pseudo vertebrae of FIGS. 5, 15, 22, 23, 31 and 32;

FIG. 35 is an elevated perspective view, rotated and in similar scale, of a variation of the bone graft device assemblies of FIGS. 3, 4, 8, and 34;

FIG. 36 is an elevated perspective view, in reduced scale, of a variation of the bone graft device assembly of FIG. 35; and

FIG. 37 is an elevated perspective view, in reduced scale, of a variation of the bone graft device assembly of FIGS. 35 and 36.

Also, in the various figures and drawings, various reference symbols and letters are used to identify significant features, dimensions, objects, and arrangements of elements described herein below in connection with the several figures and illustrations.

### DESCRIPTION OF THE PREFERRED EMBODIMENTS

In pushing the state of the art into heretofore uncharted territory, the bone graft device according to the principles of the instant invention establishes many new possible graft configurations and capabilities that are entirely absent from the many prior art attempts at improvements. The medical practitioner that employs any of the many contemplated embodiments, modifications, and variations of the bone graft devices of the instant invention can now more than ever focus on the complex and high-precision tasks before him or her without the need to expend substantial resources and valuable time on preparing, modifying, and attempting to overcome the deficiencies of prior bone graft devices. What is possible with the device according to the principles of the instant invention is a bone graft device that is more readily capable of rapid assimilation into and fusing with the host bone tissues, and which is also capable of rendering immediate and permanent structural support upon implantation. Each of these benefits can thus speed recovery and minimize the pain and discomfort otherwise likely to be experienced in various bone graft procedures.

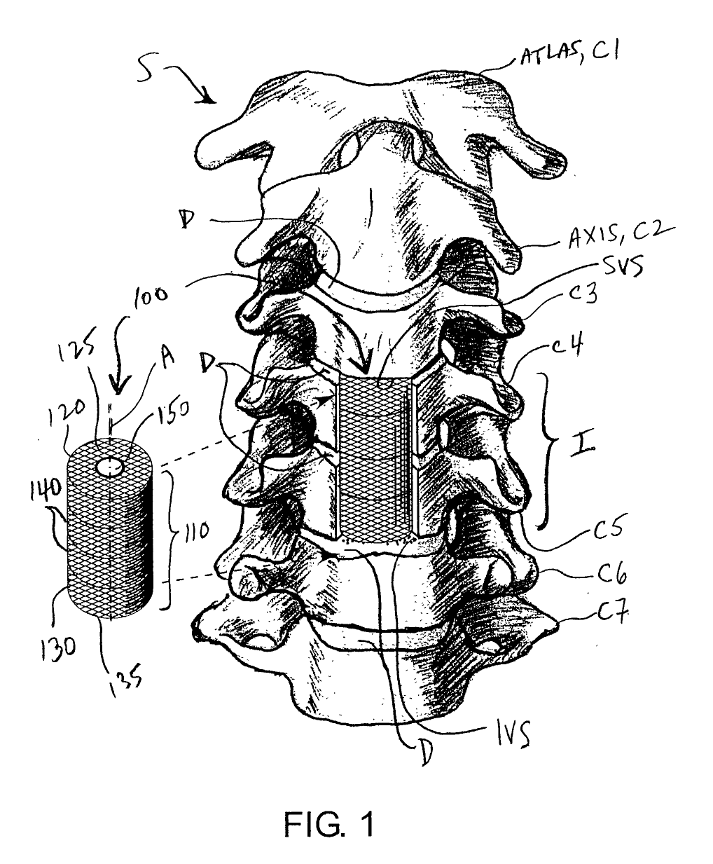

With reference now to the various figures and especially FIGS. 1, 2, 3, and 4, those having knowledge in the relevant arts may understand that the preferred bone graft device 100 according to the principles of the present inventive technology incorporates an assembly of innovative components and elements. In practice, the instant invention is contemplated and susceptible for use in a number of possible procedures and implant sites that can include cervical, thoracic, and lumbar spinal column locations. More particularly and for purposes of example without limitation, the bone graft device 100 is depicted in FIG. 1 as being assembled to be compatible for implant into a corpectomy-established anteriorly approached implant site I that is prepared and resected to span three cervical vertebral discs D and two vertebral bodies C4, C5 of a cervical spinal column S, whereby the implant site I is defined or bounded to have a caudal or an inferior vertebral surface IVS confronting a superior or rostral or cranial vertebral surface SVS. The vertebral surfaces IVS and SVS are often also referred to by those skilled in the various arts, among other surgically colloquial nomenclatures, as end plates. The surfaces IVS and SVS can be prepared to be substantially parallel or non-parallel and or to be generally concave and or convex whereby only the least amount of damaged or injured tissue is removed so as to prepare the implant site I to have the maximum amount of remaining live bone tissue. For purposes of example without limitation, the proposed exemplary corpectomy procedure can achieve arthrodesis or fusion of the bone implant graft device 100 into the host tissue of the spinal column S as healing and recovery advance in the normal course to accomplish decompression of the cervical column and the various nerve roots and enervated structures so as to reduce pain and discomfort that may be experienced by the patient from stenotic conditions or other anomalies.

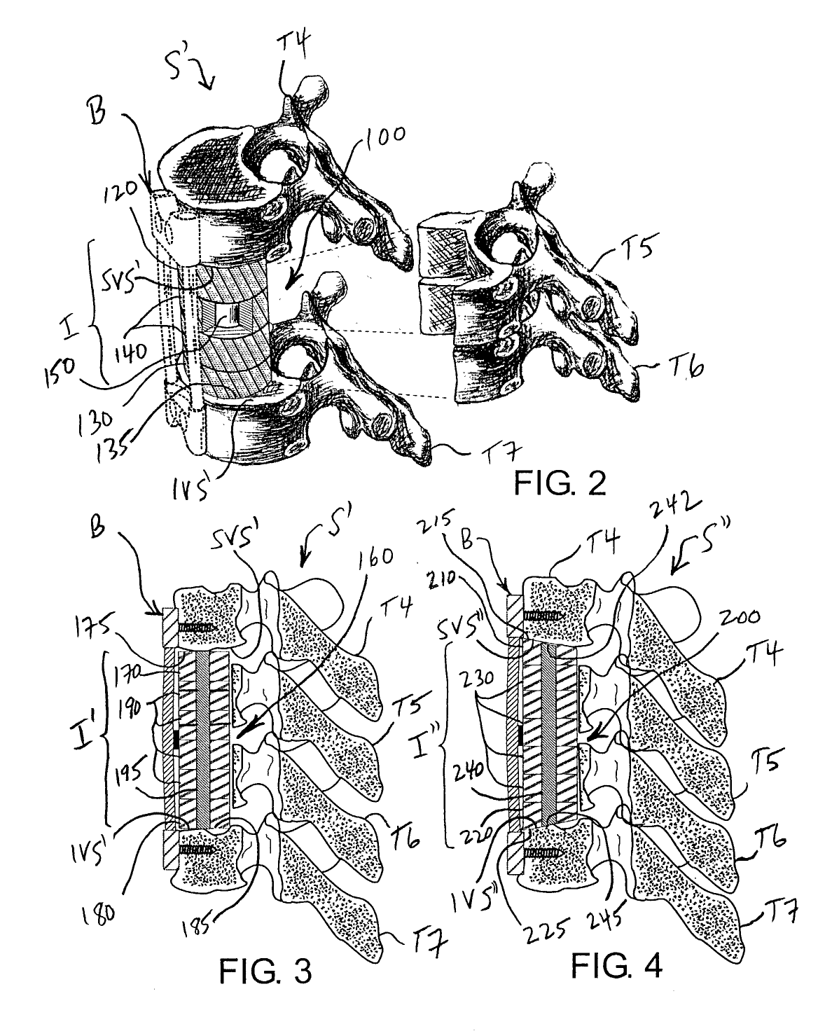

The bone graft device 100 of FIG. 1 (for an example and illustrative cervical spinal procedure) and FIG. 2 (for an illustrative thoracic or dorsal spinal procedure) includes a generally stacked arrangement of pseudo-vertebrae 110 that can be adhesively joined or bound together or be otherwise stacked. The contemplated pseudo-vertebrae arrangement 110 can include, among other elements and features, a generally cranially or rostrally or superiorly positioned abacus pseudo-vertebra 120 and a generally caudally or inferiorly positioned plinth pseudo-vertebra 130 that together cooperate to sandwich one or more drum pseudo-vertebrae 140 when the pseudo-vertebrae 120, 130, 140 are received on a strut or stanchion 150. The strut or stanchion 150 can be adapted to improve structural strength, rigidity, and alignment of the stacked pseudo-vertebrae 120, 130, 140, among other possible features and capabilities. The strut or stanchion 150 extends generally superiorly to inferiorly or rostrally to caudally though or about the pseudo-vertebrae 120, 130, 140 and about an axis denoted generally by reference letter A. The abacus and plinth pseudo-vertebrae 120, 130 incorporate at least one exteriorly or outwardly facing respective sills 125, 135 that confront and are respectively received against the corresponding vertebral surfaces SVS, ISV (and in FIG. 2, SVS′, IVS′).

For purposes of further illustration, an additional brace or stabilization instrument B that has been constructed and implanted is depicted with phantom lines in FIGS. 2, 3, and 4 as having been installed to the anterior or ventral portion of the various vertebral bodies (which could be installed in FIG. 1 on vertebrae C3 and C6 and which is shown in FIGS. 2, 3, and 4 installed on, for example without limitation, T4 and T7) for added support. For improved illustration purposes, in FIG. 2 the exemplary T5 and T6 have been extended away along the depicted dashed extension lines from their respective actual positions in the thoracic or dorsal spinal column as reflected in FIGS. 3 and 4. In the configurations of the bone graft device 100 of FIGS. 1 and 2, the exteriorly or outwardly facing respective sills 125, 135 and the respectively corresponding and confronting vertebral surfaces SVS, SVS′ and IVS, IVS′ are adapted to be substantially parallel across the respective implant sites I, I′. However, as further described hereinbelow, the instant invention is also well-suited for purposes of in applications where the respective sills 125, 135 and the respectively corresponding and confronting vertebral surfaces SVS, SVS′ and IVS, IVS′ are askew and substantially non-parallel.

In FIG. 3, which also depicts the noted exemplary thoracic or dorsal spinal corpectomy resection and graft implant procedure, an alternative bone graft device 160 is shown installed in the implant site I′ of the spinal column S′. The bone graft device 160 is similarly constructed to have a rostral or an abacus pseudo-vertebra 170 having a sill 175 received against superior or rostral vertebral surface SVS′ and a caudal or a plinth pseudo-vertebra 180 with a sill 185 received against inferior or caudal vertebral surface IVS′. The vertebral surfaces SVS′ and IVS′ are depicted having been adapted to be substantially parallel, and the corresponding outwardly or exteriorly facing sills 175, 185 are generally similarly configured.

The abacus and plinth pseudo-vertebrae 170, 180 cooperate to sandwich a plurality of drum pseudo-vertebrae 190 in a stacked arrangement that is received upon strut or stanchion 195. A different number of pseudo-vertebrae 170, 180, 190 are incorporated in this graft device 160 so as to accommodate compatibility with the differently dimensioned implant site I′. The various figures illustrate schematically proportioned pseudo-vertebrae, such as pseudo-vertebrae 120, 130, 140, 170, 180, 190, to have similar shapes, profiles, and dimensions. However, the instant invention contemplates that such pseudo-vertebrae will have a variety of different shapes, dimensions, thicknesses, and cross-sectional profiles.

Such multiply configured pseudo-vertebrae can then be selected by the medical practitioner, once the implant sites, such as sites I, I′, have been prepared, so that an optimum fitting bone graft device 100, 160 can be arranged for a net or interference fit into the implant site I, I′ to optimize structural support of the implanted bone graft device 100, 160. For example, those skilled in the related arts should be able to comprehend that the pseudo-vertebrae 170, 180, 190, can each have varied superior to inferior thicknesses so that the bone graft device 160 can be “shimmed” or otherwise sized about its gross top to bottom or superior to inferior dimension between sills 175 and 185 to fit into the implant site I′ with a net fit or interference fit whereby the implant will be snugly and or tightly received into the implant site I′.

In FIG. 4, another implant site I″ of a spinal column S″ is illustrated and defined between non-parallel vertebral surfaces SVS″ and IVS″. A bone graft device 200 is configured with generally wedge and or trapezoidally-shaped abacus and plinth pseudo-vertebrae 210, 220 that have respective inclined exteriorly facing sills 215, 225 that are adapted to be received against the non-parallel vertebral surfaces SVS″ and IVS″ to maximize contact there between. The medical practitioner in this instant has selected abacus and plinth pseudo-vertebrae 210, 220 to have suitably thick and cross-sectionally (about a sagittal or coronal cutting plane) shaped and dimensioned profiles so the contact interface between the exteriorly facing sills 215, 225 and the non-parallel vertebral surfaces SVS″ and IVS″ is maximized. In this way, the load path through the spinal column S″ and the bone graft implant device 200 is optimized to offer the most possible post-operative or post-implant structural support. The abacus and plinth pseudo-vertebrae 210, 220 sandwich drum pseudo-vertebrae 230 and all pseudo vertebrae 210, 200, 230 are received about at least one strut or stanchion 240. The strut or stanchion 245 can be further optionally or preferably formed with generally non-parallel inclined ends 242, 245 for further improved interfacing with the vertebral surfaces SVS″, IVS″.

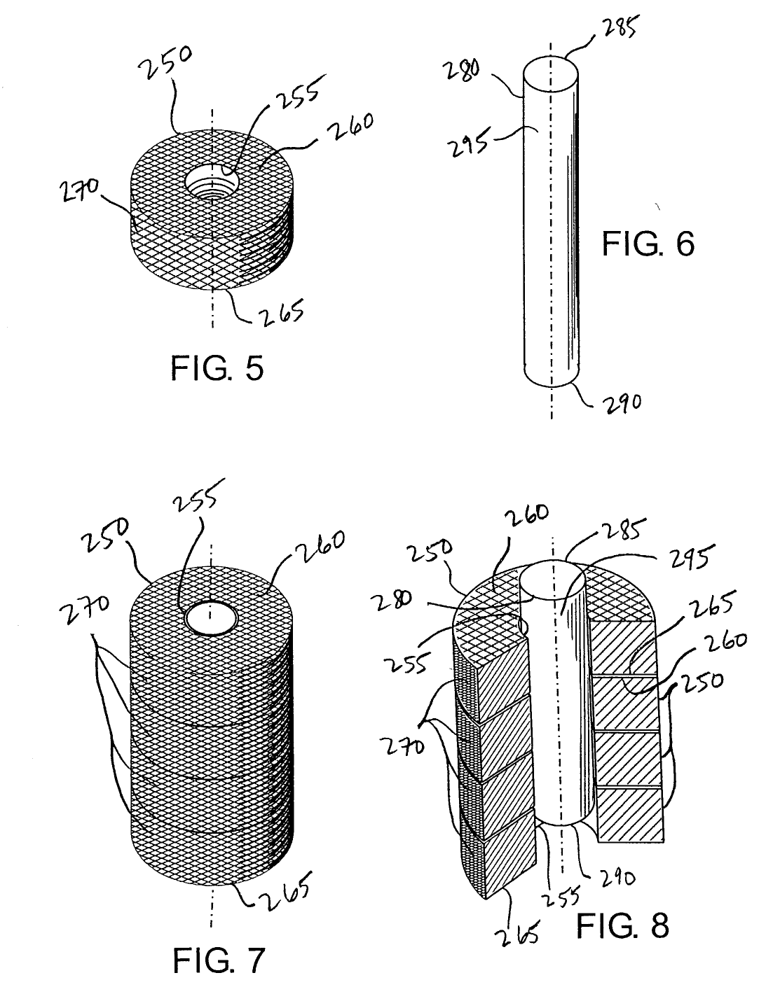

With continued reference to the various figures and specifically now also to FIGS. 5, 6, 7, an 8, further details of the devices and components of the instant invention can be understood. A pseudo-vertebra 250, which is schematically similar in construction in some aspects to the pseudo-vertebrae of bone graft devices 100, 160, 200, is depicted and has a generally centrally or medially positioned aperture 255. The aperture 255 can be positioned as shown or in any of a number of other equally suitable optional or preferable configurations that can better accommodate compatibility for use in a variety of implant site arrangements. The proposed aperture 255 can be incorporated for purposes of establishing a partial or through channel that can support and promote vascularization and or deposition of new bone tissue as fusion progresses. The aperture 255 can also serve as a recess for receiving a strut or stanchion or other stacking or alignment or keying elements. Additionally, the aperture 255 can be employed for both such capabilities and can be treated to have a surface roughness or smoothness or both along different portions, to have an elongated or through keyway or slot for alignment, and or to have a coating that can promote more rapid bone deposition and or ossification.

The pseudo-vertebra 250 can be generally cylindrically shaped and can also have any of a number of other possible shapes, thicknesses, wedged cross-sections, and the like as further disclosed elsewhere herein. Moreover, although shown as a substantially single material construction, the pseudo-vertebra 250 can be formed from one or more cancellous or cortical auto-, iso-, or allograft bone tissues, and or can be formed from any of a wide range of artificial materials that can include glass, ceramic, metal, composite, fiber, polymer, and other biocompatible materials, and alloys, hybrids, and combinations and compositions thereof. Among many other possible combinations, a particularly capable arrangement of the variously configured pseudo-vertebrae described herein, including for purposes of illustration but not for purposes of limitation pseudo-vertebrae 250, can be preferably or optionally adapted to be formed from a densified, compressed, or otherwise treated cancellous bone material to have improved load bearing, structural, fusion, and related properties, capabilities, and characteristics.

Additionally, wherein the various strut or stanchion configurations illustrated herein can be formed from any of a variety of the materials described in connection therewith and from any of the materials described in connection with the pseudo-vertebrae, for purposes of example only and not for purposes of limitation the contemplated struts and stanchions, such as struts and stanchions 150, can be formed from a cortical bone or other structurally similar and capable material that can be treated in any number of ways so as to improve its structural and fusion or arthrodesis capabilities and characteristics. Another suitable capable material that is useful for construction of the contemplated struts and stanchions can be formed from any number of possible polymerics and carbon fiber composites and alloys, hybrids, and compositions thereof, which materials can be adapted to closely match the structural properties of the native skeletal bones and structures or to be especially compatible for use in bone graft devices that are to be employed in the noted load sharing configurations.

In the contemplated construction of bone graft device 100 wherein the optional or preferred pseudo-vertebrae are formed from a substantially compressed cancellous bone material and the optional or preferred struts or stanchions are formed from a stronger material such as a cortical bone or a compatible non-biological material, especially efficacious load sharing can result in certain configurations after the device 100 has been implanted whereby the fusion process can be accelerated under certain circumstances. As may be known to those skilled in the various related arts, during the post-implant fusion process, the deposition of new bone tissue can at a biological molecular level be a direct function of the structurally induced stress loading upon the implanted and native structures in a given bone graft implant site. Accordingly, in circumstances where the implanted bone graft or other device bears little to no stress loading, a probability can exist wherein fusion and integration of the implanted graft and or device occurs very slowly if at all. This effect can be especially pronounced in applications wherein a brace or stabilization device, such as brace B (FIGS. 2, 3) is employed to bear the majority of the structural skeletal loads in combination with a bone graft implant such as bone graft device 100. Attempts to ensure that the skeletal forces and loads are shared between such a brace or stabilizer and the implanted bone graft have included developments in brace technology wherein the practitioner can effect what has been referred to in the art as a dynamism parameter in the brace construct that allows some range of motion or degree of freedom in the braced native skeletal structures such as the native vertebrae so that the implanted bone graft, such as bone graft device 100 is subjected to loading in the ordinary course of movement of the body of the patient. In this way, the brace B shares a part of the stresses and loads with the implanted bone graft, which can under certain circumstances improve the fusion process of the implanted graft. Such load sharing concerns can also have an important role as to the various components of the contemplated bone graft device 100.

More particularly, wherein the device 100 is selected to have a combination of cortical and cancellous materials, the good fusion results have been experienced in various procedures where the overall structural properties of the implanted bone graft device are identical to or similar to that of the native skeletal structures. Even more specifically, for purposes of example without limitation, among other useful properties and considerations, good fusion results have been obtained with bone graft devices that have been adapted such that the resultant structural modulus of elasticity and the flexure of the proposed bone graft devices is identical to or substantially or approximately the same as that of the native bone and skeletal structures that are being repaired, reconstructed, rehabilitated, and otherwise modified as part of the graft procedure. Additionally, in hybrid applications where a combination of cancellous and or cortical bone materials are used in combination with non-biological materials, including for example without limitation, polymerics and carbon fiber materials, good results are similarly obtained when load sharing is implemented and the hybrid and or composite bone graft device is adapted to have identical or substantially or approximately similar structural properties, characteristics, and capabilities, such as identical or approximately or substantially similar flexure and or modulus of elasticity, among other possibly desirable similarities. In such additionally contemplated load sharing hybrid bone graft applications, any of the contemplated pseudo-vertebrae, struts, stanchions, and other components can be formed from the noted materials.

Additionally, pseudo-vertebra 250 as well as any of the other components of the proposed bone graft device 100 and the variations, modifications, and alternative configurations thereof, can incorporate an exterior coating about transverse surfaces 260, 265 (which can be the vertebral surface interfacing sills described elsewhere herein) and or about exterior surface 270. For embodiments formed from composite and hybrid compositions of such materials and or non-biological materials, the contemplated coatings and treatments can be incorporated into the substrates during formulation and or during fabrication into the components described herein. Such treatments and coatings can include a cancellous and or cortical bone chip, powdered, paste, liquid, or other material that can also include various other substances, such as growth factors and the like, which materials and substances can be adapted to further stimulate assimilation and fusion into the host implant site and with other components and elements of the contemplated bone graft devices. Additionally contemplated and potentially efficacious treatments and coatings can include, for purposes of further example but not for purposes of limitation, demineralization, antitumor and chemotherapeutic agents, antibiotics, bactericides, fungicides, and other similarly capable biocides, as well as a range of possibly suitable osteobiologics that can control, limit, protect, and or promote or augment the implant site environment and constituents therein as well as being able to control, limit, and or promote the growth of tissues. Such coatings and treatments can be adapted for immediate and or time release into the implant site from the bone graft either through leeching or as the bone material of any such graft is absorbed and regenerated, and can be especially useful in various procedures and in certain patients not only for improving probabilities for successful fusion and arthrodesis, but can also and or concurrently be useful for limiting, controlling, and possibly eliminating infections, reinfections, and growth rates of regenerated tissues and the like.

In FIG. 6, a strut or stanchion 280 is illustrated that can be similar in construction in many respects to any of the other struts and or stanchions described elsewhere herein, including for example struts or stanchions 150, 195, 240. The strut or stanchion 280 can further be defined by rostral or superior end 285 and a caudal or inferior end 290 and with an outer surface 295. The strut or stanchion 280 can be fabricated from any of the aforementioned materials and can be treated and or coated in any manner similar to that already described in connection with the possible embodiments of the variously contemplated pseudo-vertebrae 250 illustrated herein above and below. Although a substantially elongated cylindrical profile is schematically represented in the depiction of strut or stanchion 280, a variety of other equally suitable shapes and configurations is contemplated and should be understood with reference to the other figures and descriptions herein. The general views of FIGS. 7 and 8 depict various constructions of bone graft devices in different stages of assembly that are fabricated in general from the pseudo-vertebra 250 and the strut or stanchion 280 of FIGS. 5 and 6.

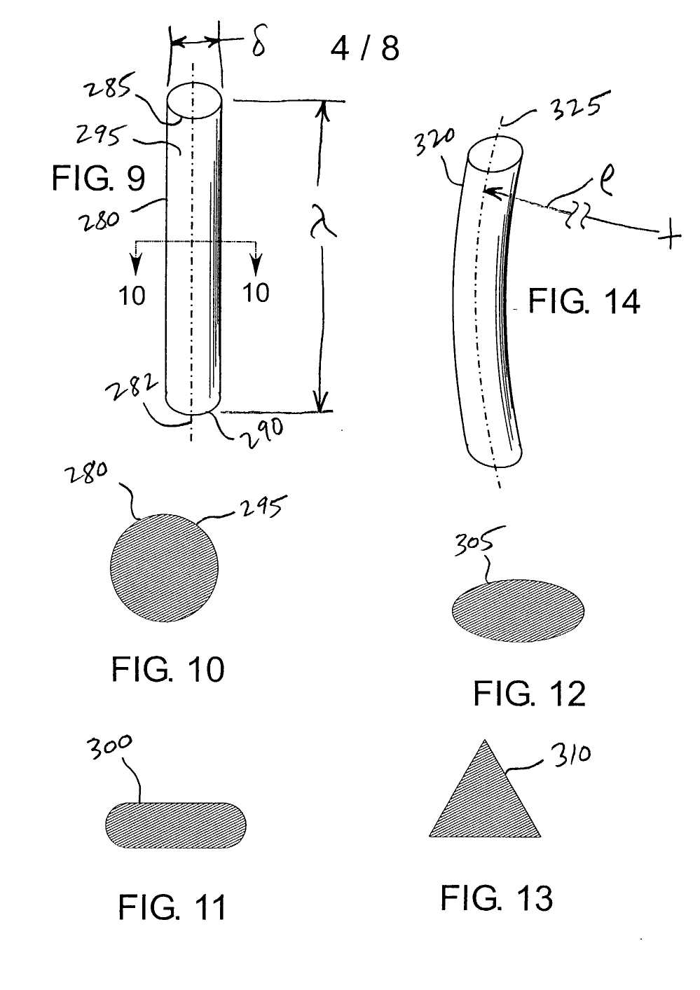

With continued reference to the various preceding illustrations and now also to FIGS. 9 through 13, further capabilities and features of the instant invention can be understood. In FIG. 9, the stanchion or strut 280 of FIG. 6 is shown for purposes of explicating additional details of contemplated constructions and parameters. Here again, although the demonstrative illustrations of FIG. 9 and the generally transverse cross-section of FIG. 10 reflect a generally cylindrical configuration, any of a number of other configurations are suitable, including the ellipsoid configuration 300 of FIG. 11, the ovoid configuration 305 of FIG. 12, and the triangular profile 310 of FIG. 13. The instant invention contemplates such struts and stanchions for use in wide range of possible bone graft implant sites and the various parameters, configurations, and arrangement of such sites will establish preferred dimensions and parameters of the most desirably strut and stanchion. In the exemplary and illustrative but non-limiting context of spinal column rehabilitative procedures, such as corpectomies, a range of suitable dimensions and parameters have been identified that can be particularly well-suited for purposes of the instant invention. While the struts and stanchions illustrated herein can be sized and shaped to substantially fill the corresponding apertures of the contemplated pseudo-vertebrae or other bone graft element adapted for use with other non-spinal corporeal bone implant sites, such struts and stanchions and struts may also be substantially undersized so as to establish an interstice between the exterior surface of the strut or stanchion and the inside surface of the aperture, which surfaces of the proposed interstice can be treated, coated, or otherwise configured to promote interstitial vascularization and bone deposition.

More particularly, the stanchion of strut 280 for use in such procedures can have a range of optional or even preferable diameters “δ” (Greek letter delta) that can preferably be in the range of about 2 to 10 millimeters, and more preferably between about 2 and 7 millimeters, and even more preferably between approximately 3 and 5 millimeters. A preferred or optional range of lengths “λ” (Greek letter lambda) will depend upon the mean average distance between the confronting vertebral surfaces, such as surfaces SVS, ISV and how large of a graft is to be implanted. In the context of spinal column procedures, the length of the strut or stanchion 280 will be a function of many levels of vertebral bodies and discs are to be spanned by the bone graft device, and the relative conditions and dimensions of the vertebral bodies and discs that exist in the operative location to be addressed by the procedure. In the cervical region of the contemplated spinal column, and subject to further modification as a result of any pronounced stenotic or other degenerative and traumatic conditions that may be present, the mean average longitudinal span across a sagittal or coronal cutting plane per cervical level can range between about 20 and 25 millimeters per vertebral body and approximately between 2 and 5 millimeters per vertebral disc. In the continued context of spinal column graft procedures, the lumbar region can present per level spans that range about 40 millimeters per vertebral body and between about 2 and 12 millimeters per vertebral disc. The thoracic region presents vertebral body and disc longitudinal spans between those of the cervical and lumbar regions of the spinal column. With these considerations in mind, the contemplated strut or stanchions 150, 195, 240, 280 could therefore preferably have a two-cervical level dimension λ in the range of between about 46 and 65 millimeters, which depend not only on the indigenous structural geometry of the spinal column to be rehabilitated, but also upon the precise parameters of the implant site established as the injured tissues are resected prior to implanting the proposed bone graft device according to the principles of the instant invention.

With reference now also specifically to FIG. 14, various other possible preferable or optional alternative arrangements are suggested that can include a strut or stanchion 320 that is adapted to have a simple or compound curvature 325 that can have a simple radius “ρ” that can be selected to specifically accommodate any native curvature present at the proposed implant site, if any. In the context of the exemplary spinal column procedures, such curvature 325 and radius ρ can be selected to approximate the particular nominal and abnormal lordotic and kyphodic, or even compound scoliotic, curvature of the spinal column at the region of the proposed implant site.

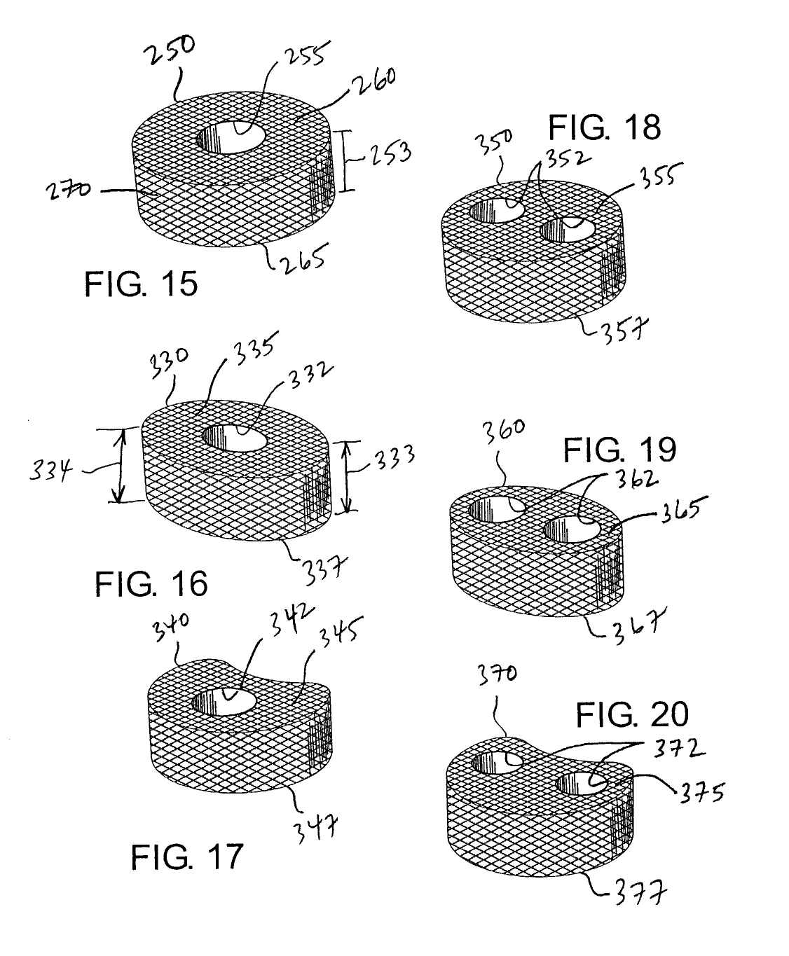

In FIGS. 15 through 21, various contemplated shapes, dimensions, and profiles of the proposed pseudo-vertebrae according to the principles of the instant invention are illustrated. With continued reference to the preceding figures and now also specifically to FIG. 15, the pseudo-vertebra 250 of FIG. 5 is represented again for comparison and further explication in the context of various other possible configurations.

The pseudo-vertebra 250 of FIG. 15 is formed to have a substantially rectilinear sagittal or coronal cross-section with a average generally uniform thickness 253 and to incorporate aperture 255 and one or more surface treatments about surfaces or sills 260, 265, which surface treatments can be substantially smooth and or include, for purposes of example without limitation, surface roughening elements that cover a portion of or all of the surfaces 260, 265. Such surface roughening features can include pointed and or diamond patterned dimples and stipples that are adapted to grip interfacing surfaces, such as the vertebral body surfaces SVS, IVS and the corresponding surfaces and or sills 260, 265 of adjacent stacked pseudo-vertebra 250 (see, e.g., FIGS. 2-4, 7, 8). Moreover, the surfaces and or sills 260, 265 can also further optionally or preferably incorporate or be coated with any of the contemplated coatings described herein elsewhere that can promote more rapid fusion and integration of the pseudo-vertebra into the host spinal column S, S′, S″.

In FIG. 16, pseudo-vertebra 330 is shown being formed with a generally trapezoidal and or wedge shaped cross section and to define what is depicted as a generally medially positioned aperture 332. The pseudo-vertebra 330 is formed with the noted wedge or trapezoidal shape to have a substantially average minimum superior to inferior thickness 333 and a substantially average maximum similar thickness 334. As with preceding embodiments, any of a variety of surface treatments and coating may be similarly incorporated. Adapted with similar thickness, surface, and coating features, another modified embodiment is illustrated by pseudo-vertebra 340 that is adapted to have an elongated or what may otherwise be referred to as a cardioid, lunular, semilunate, and or crescentric transverse or lateral cross-sectional profile, which is especially well-suited for compatibility with various of the similarly shaped vertebral bodies of the cervical, thoracic, and lumbar spinal columns.

In FIG. 18, a generally cylindrical pseudo-vertebra 350 incorporates through apertures 352 that are formed between surfaces or sills 355 and 357 and which can be cooperatively used as what can be referred to as key-ways to key or align the stacking arrangement of multiple such pseudo-vertebrae 350. Similarly, pseudo-vertebrae 360, 370 (FIGS. 19, 20) respectively incorporate through keyway apertures 362, 372 between respective superior surfaces 365, 375 and inferior surfaces 367, 377. As with preceding embodiments, configurations, and modifications, each of such pseudo-vertebrae 350, 360, 370 are easily further adapted to include any of the noted surface treatments and coatings already described in connection with other such pseudo-vertebrae.

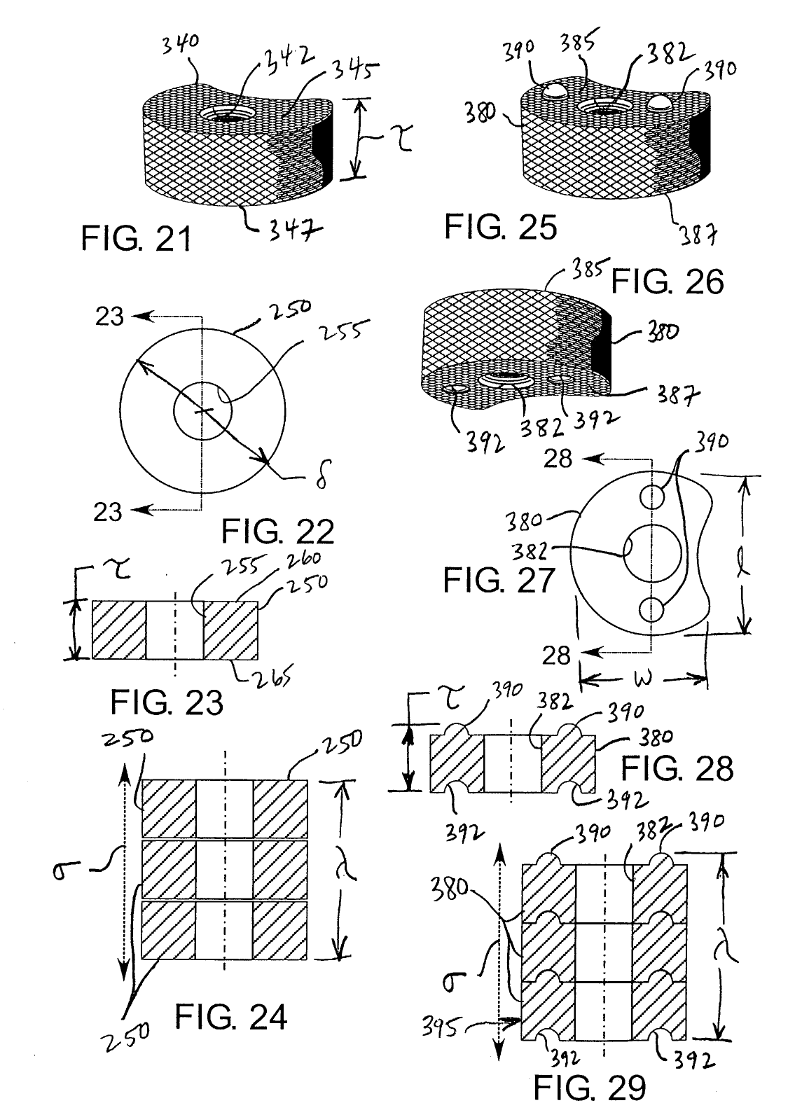

With continued reference to the preceding figures and now also specifically to FIGS. 21 through 29, the variously described pseudo-vertebrae embodiments and variations thereof are each all adapted to have various profiles as well as specific preferred and optional ranges of dimensions that are adapted to be specifically compatible for use in the respective implant sites wherein the specially configured and or assembled bone graft devices according to the principles of the instant invention are to be introduced. More specifically, and with reference now to FIG. 21, the pseudo-vertebra 340 is again depicted and is here labeled to have a thickness τ (the Greek letter tau) that can be selected to have a wide range of possible dimensions.

Typically, even though shown schematically in the various figures to have substantially similar thickness proportions, the actual preferred pseudo-vertebrae contemplated herein will be formed to have a wide range of such possible thicknesses τ that can be in the range of wafer-thin shimming configurations of about 10ths of a millimeter to sizes as large as longest span to be grafted in an adult human, which can include non-vertebral graft, rehabilitative, and reconstructive applications and procedures. In the exemplary context of spinal column grafts illustrated herein, the possible pseudo-vertebrae can be sized to span an implant site that is established across one or more levels of a large spinal lumbar vertebra and vertebral discs.

Such large nominal and healthy adult human lumbar vertebrae can be as thick about a substantially superior to inferior longitudinal or sagittal axis as between about 35 to 45 millimeters; and, such large nominal and healthy lumbar region vertebral discs can be as thick as 10 to 15 millimeters. As those skilled in the art can appreciate, such a pseudo-vertebrae thickness could thus range as high as the corresponding thickness of 2, 3, or more such lumbar levels and can be as tall as 60 to 120 millimeters or more. The thinner shimming configurations can be very well-suited for precisely configuring the proposed and contemplated bone graft devices of the instant invention.

In the generally cylindrical embodiments of the proposed and various inventive pseudo-vertebrae configurations, an average exterior generally lateral or transverse dimension is implemented to be compatible for use in the proposed graft implant site. In the context of the exemplary spinal column application described herein, the contemplated pseudo-vertebrae should be constrained to have a profile that can maximize the surface area available for transfer of loads across the implant. At the same time, the exterior profile of the proposed pseudo-vertebrae should readily fit within the confines of the pre-prepared implant site. Additionally, the pseudo-vertebrae are optimized, in the previously illustrated cardioid, semi-lunate or lunular configurations to transfer loads across as much of the interfacing vertebral body as possible while avoiding invasion of the vertebral canal or channel that contains the spinal cord nerve roots.

With specific reference to FIG. 22, the previously described pseudo-vertebra 250 is depicted to have an average generally diametrical approximate maximum dimension δ (the Greek letter delta) that is adapted to be specifically compatible with a minimum corresponding diametrical lateral or transverse dimension of an implant site, such the interfacing inferior vertebral surfaces IVS, IVS′, IVS″ and the respectively confronting superior vertebral surfaces SVS, SVS′, SVS″. While the maximum average diametrical dimension δ of the contemplated pseudo-vertebrae embodiments will depend upon whether a patient presents a nominally sized, healthy spinal columnar body at the pre-prepared implant site, such as exemplary vertebral body implant sites I, I′, I″, a variety of possible dimensions can be implemented for compatibility with the proposed implant site.

To further illustrate the possible pseudo-vertebrae dimensions δ of the spinal column procedures that contemplated for purposes of illustrating specific proposed applications, nominally health adult cervical vertebral bodies can have lateral or transverse cutting plane dimensions in the range of about 18 to 22 millimeters across a generally anterior to posterior (or ventral to dorsal) axis in the noted lateral or transverse plane, and approximately between 22 to 28 millimeters about a generally dextral to sinistral axis also in the lateral or transverse plane. In the lumbar spinal column, the corresponding approximate maximum dimensions for δ can be in the range of about 30 to 50 millimeters about the dorsal to ventral and sinistral to dextral axes spanning the transverse or lateral planes. A similar analysis and set of dimensional requirements can be discerned and applied to alternative configurations of the components that will replace the pseudo-vertebrae explicated in detail herein and which are adapted for compatibility with bone graft devices and procedures for any other part of the body that is to be addressed with any of a number of bone graft applications and procedures.

In the partially assembled schematic stacking diagrammatic representation depicted in FIG. 24 of an exemplary bone graft device, the pseudo-vertebrae, such as pseudo-vertebrae 250 are stacked in the direction generally indicated by the arrows labeled with reference letter σ (the lowercase Greek letter sigma). Any of the contemplated varied thicknesses of the contemplated pseudo-vertebrae 250 are stacked together, perhaps on a strut of stanchion or with an adhesive or binding substance (not shown), to establish a bone graft device having length λ (the lowercase Greek letter lambda) that most closely matches or nearly matches the corresponding generally longitudinal length or distance of the shortest distance between the interfacing surfaces of the implant site, such as the interfacing inferior vertebral surfaces IVS, IVS′, IVS″ and the respectively confronting superior vertebral surfaces SVS, SVS′, SVS″ already described hereinabove.

In FIGS. 25, 26, 27, 28, and 29, yet another possible key and alignment feature and capability is contemplated for use with any of the preceding embodiments, components, variations, and modifications. A pseudo-vertebra 380 can have a similar shape and construction to any of the preceding such pseudo-vertebrae to have at least one aperture 382 for receipt onto a strut of stanchion (not shown), and outwardly facing surfaces 385, 387. The proposed enhanced pseudo-vertebra 380 may also incorporate one or more keying elements such as keying stipples 390 and corresponding keyway dimples 392. If a generally cardioid or lunular profile is selected for pseudo-vertebra 380, it can be adapted in a fashion so as to maximize the load bearing surface area of surfaces 385, 387 that is available for load transfer across the pseudo-vertebra 380, while ensuring that when implanted at a spinal column implant site, the pseudo-vertebrae does not invade the spinal canal or channel, or foramen of the vertebra spanned by the assembled and implanted bone graft device. To illustrate yet another example of a specific approach to accomplish this objective, the pseudo-vertebra 380 may be formed with a transverse or lateral, sinistral to dextral dimension “l” (a lowercase Roman letter “L”) (FIG. 27) and a transverse or lateral, ventral to dorsal dimension “w” (a lowercase Roman letter “W”) (FIG. 27). In this way, a proposed alternative bone graft device 395 (FIG. 29) may be formed having multiply dimensioned thicknesses τ (FIG. 28) stacked in a direction σ to have a total bone graft device 95 stack height λ (FIG. 29) for implant into an implant site such as that already described herein.

In any of the preceding embodiments, any of the proposed and contemplated bone graft devices may have one or more such pseudo-vertebrae that can have dissimilar profiles, thicknesses, and lateral dimensions so as to establish compatibility for any of a number of possibly unusual implant site configurations. More specifically, a substantially thin, wedge or trapezoidally and cylindrically profiled pseudo-vertebra can be selected as an abacus or superior-most element, while relative thicker lunular pseudo-vertebrae may be selected and incorporated as drum or intermediate pseudo-vertebrae, while a plinth or inferior-most pseudo-vertebra having yet another shape, profile, and thickness can be incorporated so as to form a complete bone graft device that can be well-suited for a specific and peculiar implant site not otherwise expressly disclosed herein.

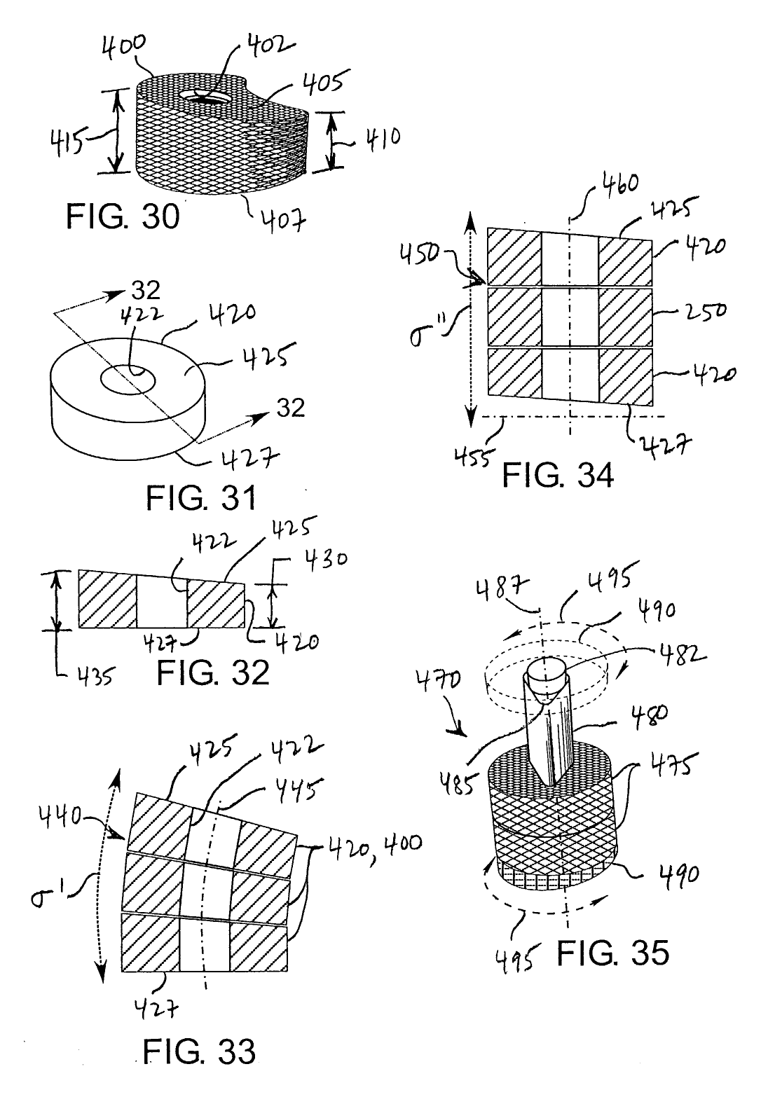

In yet other examples, in general, any of the precedingly described components and features can be incorporated in other manners so as to address other implant site and contemplated bone graft device peculiarities and objectives. More specifically, and with reference now also to FIGS. 30 to 33, another variation of a pseudo-vertebra 400 is depicted that includes an aperture 402 for stacking or other purposes, exteriorly facing surfaces or sills 405, 407, and a substantially minimum thickness 410 and a generally maximum thickness 415. The varied thicknesses 410, 415 are intended only to illustrate that a generally trapezoidal shape is implemented. However, the what may be referred to as a lofted surface that spans the surface between the minimum and maximum thickness can be planar or undulating and can establish any of a number of possible interfacing surfaces that can incorporate any of the previously described surface treatments, finishes, and coatings, as well as being adapted to interface with a specifically profiled superior or inferior graft interface surface at the implant site. More specifically, the surface or sills 405, 407 may be further shaped to also define a substantially concave or convex or other lofted surface profile that can establish automatic seating of the surfaces or sills 405, 407 against the interfacing implant site surfaces, such as inferior and superior vertebral surfaces IVS, IVS′, IVS″, SVS, SVS′, SVS″.

With specific reference to FIG. 33, such new and novel arrangements of pseudo-vertebrae, or the contemplated counterpart bone graft components adapted for use with other non-spinal corporeal graft implant sites, can be stacked to form a substantially curved bone graft device 440 that defines a curvature σ′ (FIG. 33) that can be received about a strut or stanchion (not shown) having a generally corresponding curvature 445 and which can be similar in construction in certain aspects to stanchion or strut 320 of FIG. 14. In this way, the alternative bone graft device 440 can be established to more readily match or mimic the natural lordotic or kyphodic curvature on the exemplary spinal implant sites I, I′, I″ contemplated herein as well as any other curvature indigenous to another non-spinal corporeal bone implant site.

In yet another proposed alternately configured variation, a bone implant device 450 can incorporate one or more of the previously illustrated pseudo-vertebrae such as pseudo-vertebrae 420 being positioned as the abacus superior-most and plinth inferior-most pseudo-vertebrae and stacked in direction σ″ about an intermediate drum pseudo-vertebra such as pseudo-vertebra 250. In this alternative arrangement, the exteriorly or outwardly facing surfaces 425, 427 can be established to present such surface being inclined relative to a otherwise non-inclined plane 455 that is generally orthogonal to a coronal or sagittal plane 460. This representative configuration can be readily sized for compatibility with confronting interfacing surfaces such as any of the possibly obliquely fashioned inferior and superior vertebral surfaces described elsewhere herein.

In FIG. 35, another possible bone graft device 470 is shown that can incorporate a modified drum pseudo-vertebra 475 that can be similar in some aspects to pseudo-vertebra 250 but that is modified, for non-adhesively joined graft stack arrangements wherein the intermediate pseudo-vertebrae confronting surfaces are substantially smooth, to be alignable with a keyway shaped aperture having a shape that can resist rotation, such as a substantially triangular shape, so as to ensure desired alignment of the pseudo-vertebrae remains undisturbed as the bone graft device 470 is introduced to the implant site. A strut or stanchion 480 can be also included that is received with the drum pseudo-vertebrae 475 and that is adapted with a rotation resistant cross-sectional profile that is compatible with the apertures of the pseudo-vertebrae. The strut or stanchion 480 can also further be formed with substantially cylindrical pins 482 at one or both ends that are joined or merged into corresponding pin seats 485, through which pins 482 and seats 485 passes an axis of rotation 487. Received about the pins 482 and seated against the seats 485 are additional wedge-shaped pseudo-vertebrae 490 that are adapted to rotate substantially in a plane of rotation denoted by reference arrows 495. In this configuration, the alternatively proposed bone graft device 470 can be introduced into the graft implant site whereby the rotation capable pseudo-vertebrae 490 can rotate into a more close interfacing alignment with the confronting pre-prepared surfaces of the implant site, which implant site surfaces can be non-parallel or somewhat oblique. This arrangement further establishes an even more closely aligned and more perfectly fitted implanted bone graft device 470, which in turn improves load bearing paths across the rehabilitated structure and which can further accelerate the deposition of new bone and ensuing fusion of the implant into the host tissue.

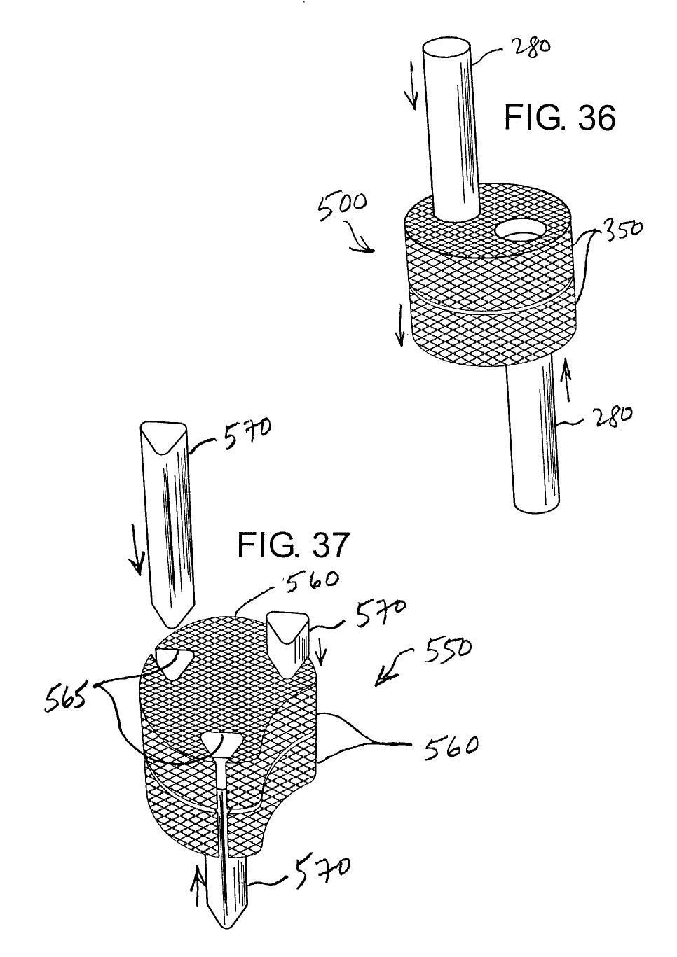

In FIG. 36, a partially assembled bone graft embodiment representative of the instant invention is depicted in bone graft device 500, which device 500 can include any of the previously described components, elements, and features. The device 500 incorporates the pseudo-vertebra 350 that are received on one or more stanchions or struts 280, which pseudo vertebrae 350 are stacked together in an aligned relationship to establish the bone graft device 500 sized to be implanted at a selected graft implant site such as any of those noted herein elsewhere. In FIG. 37, another partially assembled bone graft device 550 according to the principles of the instant invention is illustrated and includes one or more pseudo-vertebrae 560 adapted with substantially peripherally positioned keyway apertures 565 that are adapted to receive compatibly configured struts or stanchions 570. In this device 550, substantially higher load bearing capabilities can be established for possible use in very high load bone graft implant sites such as, for purposes of example without limitation, legs, arms, and lumbar spine regions of the body.

Numerous alterations, modifications, and variations of the preferred embodiments disclosed herein would be apparent to those skilled in the art and they are all contemplated to be within the spirit and scope of the instant invention, which is limited only by the following claims. For example, although specific embodiments have been described in detail, those with skill in the art can understand that the preceding embodiments and variations can be modified to incorporate various types of substitute and/or additional materials, relative arrangement of elements, and dimensional configurations for compatibility with the wide variety of possible bone graft devices and kits that are available in the marketplace. Accordingly, even though only few embodiments, alternatives, variations, and modifications of the present invention are described herein, it is to be understood that the practice of such additional modifications and variations and the equivalents thereof, are within the spirit and scope of the invention as defined in the following claims.

I claim:

1. A bone graft device adapted to be received in an anterior spinal resection of one or more vertebrae of a spinal column, the resection being defined between an inferior vertebral surface confronting a superior vertebral surface, each surface being bounded by a respective periphery defining the extents of the respective area of the surface, comprising:
a plurality of pseudo-vertebrae adapted with a cross-sectional profile compatible for implant in the anterior spinal resection;
at least one stanchion receiving the plurality of pseudo-vertebrae in a generally stacked arrangement;
wherein at least one of the plurality of pseudo-vertebrae defines an exteriorly facing sill configured to be received against at least one of the inferior and superior vertebral surfaces to maximize contact between the surfaces; and
wherein each sill is sized and shaped to be substantially circumscribed by at least one of the respective vertebral surface peripheries when received there against so as to not invade the vertebral channel of the spinal column when the bone graft device is introduced into and received in the resection.

2. The bone graft device according to claim 1, wherein at least one of the plurality of pseudo-vertebrae has a generally elongated cardioid cross-sectional profile proximal to the sill that is substantially circumscribed by the proximate vertebral surface.

3. The bone graft device according to claim 2, wherein the proximal vertebral surface is formed in a cervical vertebral body.

4. The bone graft device according to claim 2, wherein the proximal vertebral surface is formed in a thoracic vertebral body.

5. The bone graft device according to claim 2, wherein the proximal vertebral surface is formed in a lumbar vertebral body.

6. The bone graft device according to claim 1, wherein at least one of the plurality of pseudo-vertebrae is formed to have a substantially wedged shaped cross section about a sagittal plane.

7. The bone graft device according to claim 6, wherein the at least one pseudo-vertebra is adapted to rotate, as the bone graft device is introduced into the resection, about an axis substantially parallel with an axis of the at least one stanchion.

8. The bone graft device according to claim 1, wherein the at least one stanchion is at least partially curved about an axis passing longitudinally therethrough.

9. The bone graft device according to claim 1, wherein the pseudo-vertebrae are received about at least two stanchions.

10. The bone graft device according to claim 1, wherein the at least one stanchion is substantially medially disposed about the plurality of pseudo-vertebrae.

11. The bone graft device according to claim 1, wherein the at least one stanchion is substantially peripherally disposed about the plurality of pseudo-vertebrae.

12. A bone graft device adapted to be received in an anterior spinal resection of one or more vertebrae of a spinal column, the resection being defined between an inferior vertebral surface obliquely confronting a superior vertebral surface, each surface being bounded by a respective periphery defining the extents of the respective area of the surface, comprising:
at least three pseudo-vertebrae each adapted with a cross-sectional profile compatible for implant in the anterior spinal resection and further including (1) abacus and plinth pseudo-vertebra each having a substantially wedged shaped sagittal cross section and respective exteriorly facing sills adapted to maximize contact with the respective superior and inferior vertebral surfaces, and (2) at least one drum pseudo-vertebra stacked between the abacus and plinth pseudo-vertebrae;
at least one stanchion receiving the plurality of pseudo-vertebrae in a generally stacked arrangement;
wherein at least one of the abacus and plinth pseudo-vertebrae are rotatably received about the at least one stanchion; and
wherein each sill is sized and shaped to be substantially circumscribed by at least one of the respective vertebral surface peripheries when received there against so as to not invade the vertebral channel of the spinal column when the bone graft device is received in the resection.

13. The bone graft device according to claim 12, wherein at least one of the plurality of pseudo-vertebrae has a generally elongated cardioid cross-sectional profile proximal to the sill that is substantially circumscribed by the proximate vertebral surface.

14. The bone graft device according to claim 13, wherein the proximal vertebral surface is formed in a cervical vertebral body.

15. The bone graft device according to claim 13, wherein the proximal vertebral surface is formed in a thoracic vertebral body.

16. The bone graft device according to claim 13, wherein the proximal vertebral surface is formed in a lumbar vertebral body.

17. The bone graft device according to claim 12, wherein the at least one stanchion is at least partially curved about an axis passing longitudinally therethrough.

18. The bone graft device according to claim 12, wherein the at least one stanchion is substantially medially disposed about the plurality of pseudo-vertebrae.

19. The bone graft device according to claim 12, wherein the at least one stanchion is substantially peripherally disposed about the plurality of pseudo-vertebrae.

20. The bone graft device according to claim 12, further including at least two drum pseudo-vertebra that are stacked between the abacus and plinth pseudo-vertebrae, and wherein the at least two drum pseudo-vertebrae incorporate at least one key element adapted to prevent relative rotation between the at least two drum pseudo-vertebrae.

21. The bone graft device according to claim 20, wherein the respective at least one key elements cooperate with at least one stanchion key element formed in the at least one stanchion and adapted to prevent relative rotation between the at least one stanchion and the at least two drum pseudo-vertebrae.

22. The bone graft device according to claim 20, wherein the at least one key element is formed in at least one of the drum pseudo-vertebrae and is adapted to engage another of the at two drum pseudo-vertebrae to prevent relative rotation there between.

23. A bone graft kit adapted to assemble a bone graft device to be received in an anterior spinal resection of one or more vertebrae of a spinal column, the resection being defined between an inferior vertebral surface confronting a superior vertebral surface, each surface being bounded by a respective periphery defining the extents of the respective area of the surface, comprising:
a plurality of pseudo-vertebrae adapted with a cross-sectional profile compatible for implant in the anterior spinal resection wherein the pseudo-vertebrae of the plurality include at least one of shim, drum, and wedge pseudo-vertebrae;
at least one stanchion receiving the plurality of pseudo-vertebrae in a generally stacked arrangement, the at least one stanchion including at least one of a cylindrical, a keyed, and a curved stanchion;
wherein at least one of the plurality of pseudo-vertebrae defines an exteriorly facing sill configured to be received against at least one of the inferior and superior vertebral surfaces to maximize contact between the surfaces; and
wherein each sill is sized and shaped to be substantially circumscribed by at least one of the respective vertebral surface peripheries when received there against so as to not invade the vertebral channel of the spinal column when the bone graft device is received in the resection.

24. The bone graft device according to claim 23, wherein at least one of the plurality of pseudo-vertebrae has a generally elongated cardioid cross-sectional profile proximal to the sill that is substantially circumscribed by the proximate vertebral surface.

25. The bone graft device according to claim 23, wherein the shim pseudo-vertebrae is adapted with a longitudinal thickness substantially less than that of drum and wedge pseudo-vertebrae.

26. The bone graft device according to claim 23, wherein the at least one wedge pseudo-vertebra is adapted to rotate about an axis substantially parallel with an axis of the at least one stanchion.

27. The bone graft device according to claim 23, wherein the at least one stanchion is at least partially curved about an axis passing longitudinally therethrough.

28. The bone graft device according to claim 23, wherein at least two drum pseudo-vertebrae are received about at least two stanchions.

29. The bone graft device according to claim 23, wherein the at least one stanchion is substantially medially disposed about the plurality of pseudo-vertebrae.

30. The bone graft device according to claim 23, wherein the at least one stanchion is substantially peripherally disposed about the plurality of pseudo-vertebrae.
